(19) United States
(12) Patent Application Publication
(10) Pub. No.: US 2021/0259961 A1
(43) Pub. Date: Aug. 26, 2021

(54) PHARMACEUTICAL COMPOSITION FOR PREVENTING AND TREATING NITM AND MEDICAL USE THEREOF

(71) Applicant: SHENYANG XiNGQi PHARMACEUTICAL CO., LTD., Shenyang (CN)

(72) Inventors: Ningli Wang, Shenyang (CN); Jidong Liu, Shenyang (CN); Lei Guo, Shenyang (CN); Qiang Yang, Shenyang (CN); Kun Gao, Shenyang (CN); Linwei Li, Shenyang (CN); Hong Gu, Shenyang (CN); Yang Sun, Shenyang (CN); Le Fu, Shenyang (CN)

(21) Appl. No.: 17/279,555

(22) PCT Filed: Nov. 5, 2018
(86) PCT No.: PCT/CN2018/113969

§ 371 Date: Mar. 24, 2021

(30) Foreign Application Priority Data
Sep. 25, 2018 (CN) 201811119899.3

(51) Int. Cl. A61K 9/00; A61K 31/46; A61P 27/10; A61K 47/38; A61K 47/36; A61K 47/02; A61K 47/18

(52) U.S. Cl. CPC A61K 9/0048; A61K 31/46; A61P 27/10; A61K 47/186; A61K 47/36; A61K 47/02; A61K 47/183; A61K 47/38

(57) ABSTRACT

The present invention belongs to the field of medicine and relates to a pharmaceutical composition for preventing and treating NITM and a medical use thereof. Specifically, the pharmaceutical composition is an ophthalmic pharmaceutical composition such as an ophthalmic preparation. Specifically, the present invention relates to a pharmaceutical composition comprising 0.001% to 0.2% of atropine or a pharmaceutically acceptable salt thereof, and one or more pharmaceutically acceptable excipients; wherein the pH value of the pharmaceutical composition is of 4.0 to 6.5; the pharmaceutical composition comprises 0.5% to 5% of a pH adjusting agent, and the pH adjusting agent is any one or more selected from sodium dihydrogen phosphate, disodium hydrogen phosphate, citric acid, citrate, boric acid and borates. The pharmaceutical composition of the present invention can effectively treat and/or prevent NITM, and thus has a good application prospect.

### TECHNICAL FIELD

The invention belongs to the field of medicine and relates to a pharmaceutical composition for preventing and treating NITM and a medical use thereof. Specifically, the pharmaceutical composition is an ophthalmic pharmaceutical composition such as an ophthalmic preparation.

### BACKGROUND ART

Myopia means that parallel light rays from a distance are focused in front of the photoreceptor layer of retina when the adjustment function is not used. In short, parallel light rays are focused in front of the retina when the eye is at rest. There is no uniform international standard that how much away from 0D the refraction is to be considered myopia. It is more practical to take −0.25D or 0.50D as the standard (see “Myopia”, edited by Hu Daning, et al, People's Medical Publishing House, First edition, June 2009, page 3).

The Ophthalmology Refractive Optics Group, Ophthalmology Branch of Chinese Medical Association formulated the “Definition and Classification Criteria for True and False Myopia” in 1985, which divided myopia into three categories: false myopia, true myopia and half-true myopia (see “Myopia”, edited by Hu Daning, et al, People's Medical Publishing House. First edition, June 2009, pages 25-26). In addition, myopia may also be divided into false myopia, true myopia and pathological myopia (see “Blue Book 2015 for Myopia Prevention and Control and Blindness Prevention Model”, edited by Li Jianjun, People's Medical Publishing House, First edition, February 2016, pages 8-9), or myopia may be divided into pseudo-myopia, true myopia and mixed myopia (see “Ophthalmology Refractive Optics”, edited by Xu Guangdi, Military Medical Science Press, First edition, June 2005, pages 71-72; “Ophthalmology and Optometry”, edited by Wang Yuliang and Li Kai, People's Military Medical Press, First edition, August 2008, pages 399-401, “Classification of myopia”).

Pseudo-myopia, also known as accommodative myopia or functional myopia, refers to a kind of myopia under normal conditions, which may disappear after using an accommodating paralysis drug (1% atropine eye drops, three times a day for three consecutive days; or once a day for seven consecutive days) and appear as emmetropia or hyperopia; after the effect of the paralysis drug disappears, myopia appears again quickly. In China, pseudo-myopia accounts for about 5% to 10% of adolescent myopia, and appears mainly as myopia with a course of less than one year and a diopter below −1.00D. It is generally believed that pseudo-myopia is the primary stage of the occurrence and development of myopia (see “Myopia”, edited by Hu Daning, et al, People's Medical Publishing House, June 2009, First edition, page 28; “Definition and Classification Criteria for True and False Myopia”, formulated by the Ophthalmology Refractive Optics Group, Ophthalmology Branch of Chinese Medical Association).

Near-work Induced Transient Myopia (NITM) is a kind of minor and temporary shifting of far point to the front of eye due to the lens's inability to quickly and effectively reduce its own refractive power after a period of near-work, it is known to involve the accommodative lag phenomenon associated with the interaction between sympathetic nerve and parasympathetic nervous systems. NITM refers to a change in diopter (spherical equivalent degree: spherical diopter+½ cylindrical diopter) after near-work, and its value is the difference between the refractive diopter after near-work and the refractive diopter before near-work (see Lin Zhong et al. Research status of transient myopia induced by near-work, Chinese Journal of Ophthalmology, July 2012, Volume 48, Issue 7, page 657).

Regarding NITM, it has not drawn enough attention yet, and there is no corresponding effective treatment or prevention method.

### CONTENTS OF THE INVENTION

After in-depth research and creative work, the inventors surprisingly found that low-concentration atropine can effectively treat NITM, and the therapeutic effect shows a dose-dependent pharmacodynamic relationship (0.001% to 0.2%). The inventors further found that the therapeutic effect is better in borate system than phosphate buffer system, and better in osmosis adjustment using polyols than using inorganic salt such as sodium chloride, and the best effect is achieved especially when only boric acid is used to adjust pH value and osmotic pressure. The pharmaceutical composition or ophthalmic preparation of the present invention may also comprise a thickener used in conventional ophthalmic preparations, preferably hydroxypropyl cellulose, and the pharmaceutical composition or ophthalmic preparation may or may not comprise a bacteriostatic agent.

Therefore, the following invention is provided:

One aspect of the present invention relates to a pharmaceutical composition, comprising atropine or a pharmaceutically acceptable salt thereof in an amount of 0.001% to 0.2%, and one or more pharmaceutically acceptable excipients;

wherein,

the pharmaceutical composition has a pH value of 4.0 to 6.5;

the pharmaceutical composition comprises a pH adjusting agent in an amount of 0.05% to 5%, and the pH adjusting agent is any of one or more selected from a group consisting of sodium dihydrogen phosphate, disodium hydrogen phosphate, citric acid, citrate, boric acid and borate.

The pH adjusting agent may be used or added in form of a solid, or used or added in form of a buffer/buffer system (e.g., phosphate buffer, citrate buffer, borate buffer, etc.).

Regarding the pH adjusting agent, it is not excluded that sodium dihydrogen phosphate, disodium hydrogen phosphate, citric acid, citrate, boric acid and/or borate may also have another effect or function.

In one or more embodiments of the present invention, the pharmaceutical composition is an ophthalmic preparation, such as an ophthalmic liquid preparation (e.g., eye drop, eye wash or intraocular injection solution), ophthalmic semi-solid preparation (e.g., eye ointment, ophthalmic cream or ophthalmic gel), or ophthalmic solid preparation (e.g., eye film, eye pill or intraocular insert); optionally, the ophthalmic liquid preparation may be packaged in solid form, in which a solvent is provided separately, and a solution or suspension is prepared before use.

In one or more embodiments of the present invention, the pharmaceutical composition, wherein the atropine or a pharmaceutically acceptable salt thereof has a concentration or content of 0.001% to 0.1%, preferably 0.005% to 0.05%, more preferably 0.005% to 0.02%, more preferably 0.005% to 0.015% or 0.008% to 0.012%, such as 0.008%, 0.009%, 0.010%, 0.011% or 0.012%, particularly preferably 0.01%; preferably, the pharmaceutically acceptable salt of atropine is atropine sulfate.

In one or more embodiments of the present invention, the pharmaceutical composition, wherein the pharmaceutical composition has a pH of 4.0 to 6.0, preferably 4.5 to 6.0, more preferably 4.5 to 5.5, such as 4.5 to 5.0, 5.5 to 5.5, 4.5, 4.6, 4.7, 4.8, 4.9, 5.0, 5.1, 5.2, 5.3, 5.4 or 5.5.

In one or more embodiments of the present invention, the pharmaceutical composition, wherein:

the pH adjusting agent has a content of 0.5% to 5%, more preferably 1% to 3%, further preferably 1.5% to 2.5%, 1.5% to 2.0%, 1.5% to 2.25% or 1.75% to 2.25%, such as 1.5%, 1.6%, 1.7%, 1.75%, 1.8%, 1.9%, 2.0%, 2.1%, 2.2%, 2.25%, 2.3%, 2.4% or 2.5%, particularly preferably 1.75% to 2.0%;

preferably, the citrate salt is sodium citrate, and/or the borate salt is sodium tetraborate.

In one or more embodiments of the present invention, the pharmaceutical composition, wherein the pharmaceutical composition comprises boric acid in an amount of 0.5% to 3%, 1% to 3%, 1.5% to 2.5%, 1.5% to 2.25%, 1.5% to 2.0%, 1.75% to 2.25% or 1.75% to 2%, such as 1.5%, 1.6%, 1.7%, 1.75%, 1.8%, 1.9%, 2.0%, 2.1%, 2.2%, 2.25%, 2.3%, 2.4% or 2.5%.

In one or more embodiments of the present invention, the pharmaceutical composition, wherein the pharmaceutical composition further comprises any one or more items selected from the following items 1) to 5) (e.g., any 2 items, 3 items, 4 items or 5 items among them):

1) a thickener, and the thickener is any one or more selected from a group consisting of hypromellose, sodium carboxymethyl cellulose and sodium hyaluronate; preferably, the thickener has a content of 0.01% to 5%, preferably 0.5% to 3%, more preferably 0.5% to 1.5%, such as 0.5%, 0.6%, 0.7%, 0.8%, 0.9%, 1.0%, 1.1%, 1.2%, 1.3%, 1.4% or 1.5%; particularly preferably 0.8% to 1.2%.

2) an osmotic pressure regulator, for example, any one or more selected from a group consisting of glycerin, mannitol, propylene glycol, sodium chloride and potassium chloride:

3) a preservative, for example, any one or more selected from a group consisting of benzalkonium chloride, parabens bacteriostatic agents, and polyquaterniums;

4) a stabilizer, for example, any one or more selected from a group consisting of disodium edetate and calcium sodium edetate;

5) an appropriate amount of water.

In one or more embodiments of the present invention, the pharmaceutical composition comprises no preservative, and/or no stabilizer.

In one or more embodiments of the present invention, the pharmaceutical composition, wherein besides the active ingredient (atropine and/or pharmaceutically acceptable salt thereof, such as atropine sulfate) and excipients, the rest is water.

In one or more embodiments of the present invention, the pharmaceutical composition, wherein the ingredients and content thereof in the pharmaceutical composition are selected from any one of the following groups (1) to (8):

| (1) | |
|---|---|
| Atropine sulfate | 0.05 to 0.15 parts by weight |
| Hypromellose | 0.5 to 1.5 parts by weight |
| Sodium hyaluronate | 0 to 0.1 parts by weight |
| Boric acid | 1.5 to 2.5 parts by weight |
| Disodium edetate | 0 to 0.1 parts by weight |
| Benzalkonium chloride | 0 to 0.01 parts by weight |

Hydrochloric acid or sodium hydroxide, added to adjust pH to 4.5 to 6.0

Water for injection, added to reach 100 parts by weight:

| (2) | |
|---|---|
| Atropine sulfate | 0.01 parts by weight |
| Hypromellose | 0.5 to 1.2 parts by weight |
| Sodium hyaluronate | 0 to 0.05 parts by weight |
| Boric acid | 1.5 to 2.0 parts by weight |
| Disodium edetate | 0.005 to 0.1 parts by weight |
| Benzalkonium chloride | 0 to 0.01 parts by weight |

Hydrochloric acid or sodium hydroxide, added to adjust pH to 4.5 to 5.5

Water for injection, added to reach 100 parts by weight;

| (3) | |
|---|---|
| Atropine sulfate | 0.01 parts by weight |
| Hypromellose | 0.8 to 1.0 parts by weight |
| Sodium hyaluronate | 0.02 to 0.05 parts by weight |
| Boric acid | 1.75 to 2.0 parts by weight |
| Disodium edetate | 0.01 to 0.05 parts by weight |
| Benzalkonium chloride | 0 to 0.01 parts by weight |

Hydrochloric acid or sodium hydroxide, added to adjust pH to 4.5 to 5.5

Water for injection, added to reach 100 parts by weight;

| (4) | | |
|---|---|---|
| Atropine sulfate | 0.010 | parts by weight |
| Hypromellose | 0.800 | parts by weight |
| Sodium hyaluronate | 0.02 | parts by weight |
| Boric acid | 2.00 | parts by weight |
| Disodium edetate | 0.005 | parts by weight |
| Benzalkonium chloride | 0 | parts by weight |

Hydrochloric acid or sodium hydroxide, added to adjust pH to 4.5

Water for injection, added to reach 100 parts by weight:

| (5) | | |
|---|---|---|
| Atropine sulfate | 0.010 | parts by weight |
| Hypromellose | 0.500 | parts by weight |
| Sodium hyaluronate | 0.05 | parts by weight |
| Boric acid | 2.00 | parts by weight |
| Disodium edetate | 0.010 | parts by weight |
| Benzalkonium chloride | 0.010 | parts by weight |

Hydrochloric acid or sodium hydroxide, added to adjust pH to 5.0

Water for injection, added to reach 100 parts by weight;

| (6) | | |
|---|---|---|
| Atropine sulfate | 0.010 | parts by weight |
| Hypromellose | 1.000 | parts by weight |
| Sodium hyaluronate | 0 | parts by weight |
| Boric acid | 1.750 | parts by weight |
| Disodium edetate | 0.050 | parts by weight |
| Benzalkonium chloride | 0 | parts by weight |

Hydrochloric acid or sodium hydroxide, added to adjust pH to 5.5

Water for injection, added to reach 100 parts by weight;

| (7) | | |
|---|---|---|
| Atropine sulfate | 0.010 | parts by weight |
| Hypromellose | 1.200 | parts by weight |
| Sodium hyaluronate | 0 | parts by weight |
| Boric acid | 2.0 | parts by weight |
| Disodium edetate | 0.010 | parts by weight |
| Benzalkonium chloride | 0.010 | parts by weight |

Hydrochloric acid or sodium hydroxide, added to adjust pH to 5.0

Water for injection, added to reach 100 parts by weight:

| (8) | | |
|---|---|---|
| Atropine sulfate | 0.010 | parts by weight |
| Hypromellose | 1.000 | parts by weight |
| Sodium hyaluronate | 0 | parts by weight |
| Boric acid | 1.750 | parts by weight |
| Disodium edetate | 0.100 | parts by weight |
| Benzalkonium chloride | 0 | parts by weight |

Hydrochloric acid or sodium hydroxide, added to adjust pH to 6.0

Water for injection, added to reach 100 parts by weight.

The pharmaceutical composition of the present invention can be prepared by technical means known to those skilled in the art.

Another aspect of the present invention relates to use of atropine or a pharmaceutically acceptable salt thereof or a pharmaceutical composition comprising atropine or a pharmaceutically acceptable salt thereof in manufacture of a medicament for treatment and/or prevention of NITM (near-work induced transient myopia);

preferably, the pharmaceutically acceptable salt is atropine sulfate;

preferably, the pharmaceutical composition further comprises one or more pharmaceutically acceptable excipients:

preferably, the pharmaceutical composition is the pharmaceutical composition of any one of the items of the present invention.

In one or more embodiments of the present invention, the use, wherein the drug is an ophthalmic preparation, preferably an eye drop.

In one or more embodiments of the present invention, the use, wherein the eye drop is administered once a day or every other day, 1 to 2 drops each time; preferably, the eye drop is dripped into a conjunctival sac.

In one or more embodiments of the present invention, the use, wherein the eye drop is administered to a subject who is a person using eyes in near distance or a person susceptible to myopia:

preferably, the person using eyes in near distance is a myopia patient or a non-myopia patient;

preferably, the person susceptible to myopia is a person aged 6 to 18.

Another aspect of the present invention relates to a packed medicament product, which comprises a medicament and a product specification;

wherein, the medicament is atropine or a pharmaceutically acceptable salt thereof, or a pharmaceutical composition comprising atropine or a pharmaceutically acceptable salt thereof;

preferably, the pharmaceutically acceptable salt is atropine sulfate;

preferably, the pharmaceutical composition further comprises one or more pharmaceutically acceptable excipients;

preferably, the pharmaceutical composition is the pharmaceutical composition of any one of the items of the present invention;

preferably, the medicament is an ophthalmic preparation, preferably an eye drop.

In one or more embodiments of the present invention, the packed medicament product, wherein the product specification states that the eye drop is administered once a day or every other day, 1 to 2 drops each time;

preferably, the product specification also states that the eye drop is dripped into a conjunctival sac.

In one or more embodiments of the present invention, the packed medicament product, wherein the product specification states that the eye drop is administered to a subject who is a myopia patient or a non-myopia patient;

preferably, the product specification states that the eye drop is administered to a subject who is a person using eyes in near distance;

preferably, the product specification states that the eye drop is administered to a subject who is a myopia patient or non-myopia patient using eyes in near distance;

preferably, the product specification states that the eye drop is administered to a subject who is a person susceptible to myopia;

preferably, the product specification states that the eye drop is administered to a subject who is a person aged 6 to 18, such as a myopia patient aged 6 to 18 or a non-myopia patient aged 6 to 18.

Another aspect of the present invention relates to atropine or a pharmaceutically acceptable salt thereof, or a pharmaceutical composition containing atropine or a pharmaceutically acceptable salt thereof, for use in the treatment and/or prevention of NITM (near-work induced transient myopia);

preferably, the pharmaceutically acceptable salt is atropine sulfate:

preferably, the pharmaceutical composition further comprises one or more pharmaceutically acceptable excipients;

preferably, the pharmaceutical composition is the pharmaceutical composition of any one of the items of the present invention.

In one or more embodiments of the present invention, the pharmaceutical composition is an ophthalmic preparation, preferably an eye drop.

In one or more embodiments of the present invention, the atropine or a pharmaceutically acceptable salt thereof, or the pharmaceutical composition containing atropine or a pharmaceutically acceptable salt thereof, wherein the eye drop is administered once a day or every other day, 1 to 2 drops each time; preferably, the eye drop is dripped into a conjunctival sac.

In one or more embodiments of the present invention, the atropine or a pharmaceutically acceptable salt thereof, or the pharmaceutical composition containing atropine or a pharmaceutically acceptable salt thereof, wherein the eye drop is administered to a subject who is a person using eyes in near distance or a person susceptible to myopia;

preferably, the person using eyes in near distance is a myopia patient or a non-myopia patient;

preferably, the person susceptible to myopia is a person aged 6 to 18.

Another aspect of the present invention relates to a method for treating and/or preventing NITM, comprising a step of administering an effective amount of a medicament to a subject in need thereof, wherein the medicament is atropine or a pharmaceutically acceptable salt thereof, or a pharmaceutical composition containing atropine or a pharmaceutically acceptable salt thereof;

preferably, the pharmaceutically acceptable salt is atropine sulfate;

preferably, the pharmaceutical composition further comprises one or more pharmaceutically acceptable excipients;

preferably, the pharmaceutical composition is the pharmaceutical composition according to any one of the items of the present invention;

preferably, the pharmaceutical composition is an ophthalmic preparation, preferably an eye drop.

In one or more embodiments of the present invention, the method for treating and/or preventing NITM, wherein the eye drop is administered once a day or every other day, 1 to 2 drops each time; preferably before sleep;

preferably, the eye drop is dripped into a conjunctival sac.

In one or more embodiments of the present invention, the method for treating and/or preventing NITM, wherein the eye drop is administered to a subject who is a myopia patient or a non-myopia patient;

preferably, the eye drop is administered to a subject who is a person using eyes in near distance;

preferably, the eye drop is administered to a subject who is a myopia patient or non-myopia patient using eyes in near distance;

preferably, the eye drop is administered to a subject who is a person susceptible to myopia, such as a person aged 6 to 18;

preferably, the person susceptible to myopia is a myopia patient or a non-myopia patient.

The actual dosage and period of administration can be determined according to the subject's own physical conditions or in accordance with the doctor's advice. Under normal circumstances, it can be administered in a short or long term, such as 1 month, 2 months, 3 months, 4 months, 5 months, 6 months, 1 year, 1.5 years, 2 years, 2.5 years, 3 years or more than 3 years.

Some terms involved in the present invention are explained as follows:

The structural formula of atropine is shown in the following formula I:

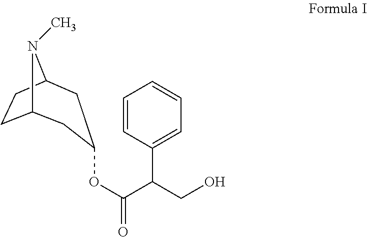

The structure of atropine sulfate is shown in the following formula II:

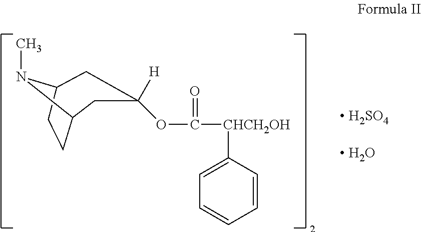

In the present invention, the term “person using eyes in near distance” refers to a person whose eyes are relatively close to a gaze target, and who maintains continuous gaze for a period of time. In a narrow sense: for the term “person using eyes in near distance”, “near distance” or “shorter distance” means that the straight line distance between the eyes and the gaze target is 10 cm to 50 cm; “a period of time” refers to at least 30 minutes, at least 45 minutes, at least 1 hour, at least 1.5 hours, at least 2 hours, at least 2.5 hours, at least 3 hours, at least 4 hours, or more than 4 hours, wherein “continuous gaze” refers to uninterrupted gaze; in some individual cases, one, two or three interruptions may be allowed, but each interruption time does not exceed 5 seconds, preferably not more than 3 seconds or not more than 2 seconds. Generally, the person using eyes in near distance includes but is not limited to elementary and middle school students, office white-collar workers, persons engaged in electronic games or videos, etc.; the gaze target includes, but is not limited to, electronic display devices (mobile phone displays, computer displays, electronic readers, etc.), books, examination papers, etc. The person using eyes in near distance includes myopia patients and non-myopia patients.

In the present invention, the term “person susceptible to myopia” includes persons who are susceptible to myopia but have not yet suffered from myopia, or persons who are already suffering from myopia and are likely to further aggravate the degree of myopia. Generally speaking, persons aged 6 to 18 (children, adolescents) are susceptible to mopia because their eyes and optic nervous system are not yet fully developed, or because they are under pressure to study, or are exposed to video games or videos frequently. The person susceptible to myopia includes myopia patients and non-myopia patients, preferably myopia patients or non-myopia patients aged 6 to 18.

In the present invention, the concentration of the atropine or a pharmaceutically acceptable salt thereof (e.g., atropine sulfate), if not otherwise specified, refers to mass/volume percentage concentration. In the present invention, the content of the atropine or a pharmaceutically acceptable salt thereof (e.g., atropine sulfate), if not specifically specified, refers to mass/volume (g/ml) percentage content.

In the present invention, the content of a certain excipient material (e.g., boric acid), if not otherwise specified, refers to mass/volume (g/ml) percentage content.

The mass/volume percentage refers to the number of grams of solute contained in a volume of one hundred milliliters. The relative density of the liquid preparation involved in the present invention is about 1.0. Therefore, in the actual preparation process, it can be calculated as hundred grams per hundred milliliters. Therefore, the mass/volume (g/ml) percentage can also be approximately mass/mass (g/g) percentage.

### THE BENEFICIAL EFFECTS OF THE PRESENT INVENTION

The present invention has achieved one or more of the following technical effects:

(1) for the pharmaceutical composition of the present invention, the use of boric acid and/or borate to adjust pH and/or osmotic pressure achieves very good effect, which is better than that of other pH adjusting agents and/or osmotic pressure adjusting agents;

(2) the use of boric acid and/or borate can enhance the effect of treating and/or preventing NITM;

(3) the use of boric acid and/or borate can reduce the mydriatic side effects of atropine to a certain extent;

(4) atropine or a pharmaceutically acceptable salt thereof (e.g., atropine sulfate), or a pharmaceutical composition containing atropine or a pharmaceutically acceptable salt thereof (e.g., atropine sulfate), can effectively treat and/or prevent NITM.

### SPECIFIC MODELS FOR CARRYING OUT THE INVENTION

The embodiments of the present invention will be described in detail below in conjunction with examples, but those skilled in the art will understand that the following examples are only used to illustrate the present invention and should not be regarded as limiting the scope of the present invention. If a specific condition is not indicated in the examples, it shall be carried out in accordance with conventional condition or condition recommended by the manufacturer. The reagents or instruments used without giving their manufacturers are all conventional products that are commercially available.

### Example 1: Experiment of NITM Treatment

### 1. Experimental Drug

0.01% Atropine sulfate eye drop was prepared with 0.9% saline and adjusted the pH to 6.0 with hydrochloric acid. It was used for administration in the test group.

### 2. Experimental Method

30 Myopia patients aged 6 to 18 were selected. The selected patients who had eye diopter of −0.50DS to −5.00DS, astigmatism [symbol] −1.0DC; initial NITM value [symbol] −0.25D; normal intraocular pressure not exceeding 21 mmHg, were randomly divided into 2 groups, 15 patients in each group. The test group was dripped with the aforementioned experimental drug, and the control group was dripped with 0.9% normal saline; each group was administered once a day, 1 drop per eye each time, and the aforementioned experimental drug or 0.9% normal saline was dripped into conjunctival sac before sleep for consecutive 6 months. NITM detection was performed at scheduled time points, and the initial NITM value, NITM attenuation time, and the spherical equivalent degree of glasses worn to achieve the best corrected visual acuity were recorded. The effectiveness was determined when the initial NITM value decreased by more than or equal to 0.20D, which could be used to calculate the effective rate of drug.

The drug was determined as effective in a case when the initial NITM value after administration decreased by more than or equal to 0.20D in comparison with the initial NITM at baseline.

Effective rate=number of effective cases/number of treated patients×100%.

Wherein, NITM at baseline referred to a NITM value before administration, that was, the NITM value on the 0^{th} day, and initial NITM at baseline was the difference between the average diopter within 10 s after near-work and the diopter before near-work on the 0^{th} day before administration. The measurement method of NITM at baseline was consistent with the following NITM measurement method at each time point after administration.

NITM measurement: optometry was carried out by using a phoropter (non-cycloplegia), according to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again, and the initial NITM was recorded. The initial NITM was the difference between the diopter within 10 s after near-work and the diopter before near-work (Table 1), the NITM attenuation time referred to the time taken for the initial NITM to decrease to zero (the attenuation time reflected the speed at which the eyes returned to their daily level after transient myopia caused by near-work. Unit: seconds).

### 3. Experimental Results

The results were shown in Table 1 to Table 4 below.

TABLE 1
Effect of 0.01% atropine sulfate eye drop on initial
NITM value of eyes at different time points (x̅ ± S, n = 15)

| | Observation time (days) | | | | |
|---|---|---|---|---|---|
| Group | 0 | 30 | 60 | 90 | 180 |
| Control group | 0.38 ± 0.09 | 0.37 ± 0.09 | 0.37 ± 0.07 | 0.40 ± 0.07 | 0.35 ± 0.06 |
| Test group | 0.38 ± 0.09 | 0.30 ± 0.05 | 0.23 ± 0.06 | 0.12 ± 0.06 | 0.14 ± 0.03 |

TABLE 2
Effect of 0.01% atropine sulfate eye drop on NITM attenuation
time of eyes at different time points (x̅ ± S, n = 15)

| | Observation time (days) | | | | |
|---|---|---|---|---|---|
| Group | 0 | 30 | 60 | 90 | 180 |
| Control group | 140.8 ± 12.6 | 140.9 ± 10.8 | 143.9 ± 11.6 | 139.8 ± 11.4 | 142.8 ± 11.6 |
| Test group | 138.3 ± 13.0 | 109.7 ± 8.4 | 92.9 ± 8.5 | 72.5 ± 11.7 | 50.5 ± 10.7 |

TABLE 3
Effective rate of 0.01% atropine sulfate eye drop on
NITM of eyes at different time points (x̅ ± S, n = 15)

| | Observation time (days) | | | | |
|---|---|---|---|---|---|
| Group | 0 | 30 | 60 | 90 | 180 |
| Control group | 0 | 6.67% | 0 | 0 | 0 |
| Test group | 0 | 20.00% | 33.33% | 86.67% | 80.00% |

TABLE 4
Effect of 0.01% atropine sulfate eye drop on spherical
equivalent degree of eyes at different time points (x̅ ± S, n = 15)

| | Observation time (days) | | | | |
|---|---|---|---|---|---|
| Group | 0 | 30 | 60 | 90 | 180 |
| Control group | -2.87 ± 1.23 | -2.86 ± 1.23 | -2.92 ± 1.28 | -2.97 ± 1.22 | -3.23 ± 1.14 |
| Test group | -2.85 ± 1.25 | -2.85 ± 1.25 | -2.85 ± 1.25 | -2.91 ± 1.25 | -2.92 ± 1.26 |

Note:
Diopter is classified into spherical diopter (myopia, hyperopia.; it was myopia. in the
present invention) and cylindrical diopter (astigmatism); spherical equivalent degree spherical
diopter + ½ cylindrical diopter.

It can be seen from the data in Table 1 to Table 4 that after 6 months of continuous administration, the myopia progression of the control group was −0.36±0.13D, and the myopia progression of the test group was −0.07±0.15D. That was, it had a certain control effect on the increase of myopia diopter. However, due to the short administration period, there was no statistical difference between the control group and the test group. This was potentially consistent with the results of recent studies of low-concentration atropine in controlling the progression of myopia: the progression rate of myopia within 2 years in the Singapore ATOM1 placebo group was −1.20±0.69D, and the progression of myopia within 2 years in the 0.01% atropine group in the ATOM2 trial was −0.49±0.63D; that was, long-term administration of low-concentration atropine could control the progression of myopia.

However, in this study, the inventors unexpectedly found that 0.01% atropine had a therapeutic effect on transient myopia caused by near-work in a short time, there were significant differences (one-way analysis of variance) in terms of initial NITM value and attenuation time between the control group and the test group after 1 month of administration, the effective rate of drug basically reached a steady state after 90 days of administration (greater than 80.00%), and starting from 30 days, the effective rates of the test group and the control group were statistically different (χ² test).

### Example 2: Study on Effects and Side Effects of Different Concentrations of Atropine Sulfate Eye Drops on NITM

### 1. Experimental Drugs

By using 0.9% normal saline, atropine sulfate eye drops in concentrations of 0.001%, 0.005%, 0.01%, 0.02%, 0.05%, 0.1%, 0.2% were prepared, respectively, adjusted their pH to 5.5 with hydrochloric acid, and used for administration in the test groups.

### 2. Experimental Method

120 Myopia patients aged 6 to 18 were selected. The selected patients who had eye diopter of −0.50DS to −5.00DS, astigmatism [symbol] −1.0DC; initial NITM value [symbol] −0.25D; normal intraocular pressure not exceeding 21 mmHg were randomly divided into 8 groups, 15 patients in each group. The 7 test groups were separately dripped with the 0.001%, 0.005%, 0.01%, 0.02%, 0.05%, 0.1%, 0.2% atropine sulfate eye drops prepared with 0.9% normal saline, and the control group was dripped with 0.9% normal saline; each group was administered once a day, 1 drop per eye each time, and the aforementioned drug or 0.9% normal saline was dripped into conjunctival sac before sleep at 9 p.m. for consecutive 2 weeks. NITM detection was performed at 7 a.m. on the 0^{th} day, 7^{th} day, 14^{th} day, and the initial NITM value, NITM attenuation time and pupil diameter were recorded.

Optometry was carried out by a phoropter (non-cycloplegia), and pupil diameter was measured. According to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again.

### 3. Experimental Results

The results were shown in Table 5 to Table 7 below.

TABLE 5
Effect of atropine sulfate eye drops in different
concentrations on initial NITM value of eyes (x̅ ± S, n = 15)

| | Observation time (days) | | |
|---|---|---|---|
| Group | 0 | 7 | 14 |
| Control group | 0.42 ± 0.09 | 0.42 ± 0.12 | 0.41 ± 0.11 |
| 0.001% | 0.44 ± 0.10 | 0.32 ± 0.07 | 0.32 ± 0.07 |
| 0.005% | 0.41 ± 0.09 | 0.30 ± 0.09 | 0.25 ± 0.06 |
| 0.01% | 0.43 ± 0.10 | 0.20 ± 0.10 | 0.24 ± 0.07 |
| 0.02% | 0.44 ± 0.10 | 0.25 ± 0.08 | 0.24 ± 0.09 |
| 0.05% | 0.38 ± 0.10 | 0.24 ± 0.09 | 0.17 ± 0.06 |
| 0.1% | 0.39 ± 0.08 | 0.21 ± 0.07 | 0.16 ± 0.07 |
| 0.2% | 0.44 ± 0.11 | 0.20 ± 0.07 | 0.16 ± 0.06 |

TABLE 6
Effect of atropine sulfate eye drops in different concentrations
on NUM attenuation time of eyes (x̅ ± S, n = 15)

| | Observation time (days) | | |
|---|---|---|---|
| Group | 0 | 7 | 14 |
| Control group | 136.5 ± 7.9 | 140.6 ± 14.1 | 136.6 ± 9.2 |
| 0.001% | 142.1 ± 10.6 | 133.0 ± 14.1 | 114.7 ± 8.2 |
| 0.005% | 136.6 ± 12.7 | 117.6 ± 10.4 | 97.7 ± 9.5 |
| 0.01% | 140.3 ± 13.7 | 116.1 ± 8.6 | 97.5 ± 8.8 |
| 0.07% | 137.3 ± 12.6 | 116.0 ± 7.9 | 96.9 ± 8.0 |
| 0.05% | 141.8 ± 13.7 | 114.7 ± 10.4 | 72.5 ± 11.6 |
| 0.1% | 139.9 ± 11.0 | 114.0 ± 8.7 | 63.4 ± 9.2 |
| 0.2% | 136.0 ± 11.6 | 113.6 ± 6.8 | 67.2 ± 10.6 |

TABLE 7
Effect of atropine sulfate eye drops in different
concentrations on pupil diameter of eyes (x̅ ± S, n = 15)

| | Observation time (days) | | |
|---|---|---|---|
| Group | 0 | 7 | 14 |
| Control group | 136.5 ± 7.9 | 3.6 ± 0.97 | 3.7 ± 0.73 |
| 0.001% | 142.1 ± 10.6 | 3.9 ± 0.48 | 4.0 ± 0.44 |
| 0.005% | 136.6 ± 12.7 | 4.1 ± 0.74 | 4.1 ± 0.70 |
| 0.01% | 140.3 ± 13.7 | 4.2 ± 0.68 | 4.1 ± 0.74 |
| 0.02% | 137.3 ± 12.6 | 4.5 ± 0.74 | 4.8 ± 0.71 |
| 0.05% | 141.8 ± 13.7 | 4.7 ± 0.72 | 4.9 ± 0.66 |
| 0.1% | 139.9 ± 11.0 | 4.7 ± 0.70 | 5.1 ± 0.98 |
| 0.2% | 136.0 ± 11.6 | 5.2 ± 0.48 | 5.6 ± 1.04 |

The therapeutic effect was judged by the combination of initial NITM value and NITM attenuation time, and effectiveness was determined when there were statistical differences. Specifically, the initial NITM value was the main factor and the attenuation time was the secondary factor. The determination could be made when initial NITM value showed difference. The statistics was performed by using one-way analysis of variance, and it was considered as being of statistical difference when P<0.05.

From the data in Table 5, Table 6 and Table 7, it could be seen that low-concentration atropine had a therapeutic effect on NITM and had a dose-effect relationship; when the dose was increased to 0.02%, the enhancement of the therapeutic effect decreased, and the pupil diameter gradually showed a significant difference after dropping for 14 days.

### Example 3: Study on Effect of Atropine Sulfate Eye Drops with Different pH Values on NITM

### 1. Experimental Drugs

By using 0.9% sodium chloride solution, 0.01% atropine sulfate eye drop was prepared, and adjusted with hydrochloric acid to pH value of 4.0, 4.5, 5.0, 5.5, 6.0, 6.5, respectively, and used for administration in the test groups.

### 2. Experimental Method

105 Myopia patients aged 6 to 18 were selected. The selected patients who had eye diopter of −0.50DS to −5.00DS, astigmatism [symbol] −1.0DC; initial NITM value [symbol] −0.25D; normal [symbol] intraocular pressure not exceeding 21 mmHg were randomly divided into 7 groups, 15 patients in each group. The 6 test groups were separately dripped with the above prepared atropine sulfate eye drops (whose pH values were 4.0, 4.5, 5.0, 5.5, 6.0, 6.5, respectively), and the control group was dripped with 0.9% normal saline; each group was administered once a day, 1 drop per eye each time, and the aforementioned drug or 0.9% normal saline was dripped into conjunctival sac before sleep for consecutive 2 weeks. NITM detection was performed on the 0^{th} day, 7^{th} day, 14^{th} day, and the initial NITM value and NITM attenuation time were recorded.

Optometry was carried out by a phoropter (non-cycloplegia). According to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again.

### 3. Experimental Results

The results were shown in Table 8 to Table 9 below.

TABLE 8
Effect of atropine sulfate eye drops with different
pH values on initial NITM value of eyes (x̅ ± S, n = 15)

| | Observation time (days) | | |
|---|---|---|---|
| Group | 0 | 7 | 14 |
| Control group | 0.43 ± 0.11 | 0.43 ± 0.11 | 0.43 ± 0.11 |
| pH4.0 | 0.45 ± 0.10 | 0.31 ± 0.07 | 0.31 ± 0.08 |
| pH4.5 | 0.45 ± 0.10 | 0.26 ± 0.08 | 0.26 ± 0.10 |
| pH5.0 | 0.45 ± 0.10 | 0.74 ± 0.09 | 0.25 ± 0.10 |
| pH5.5 | 0.41 ± 0.08 | 0.72 ± 0.09 | 0.23 ± 0.07 |
| pH6.0 | 0.46 ± 0.09 | 0.34 ± 0.08 | 0.35 ± 0.07 |
| pH6.5 | 0.40 ± 0.09 | 0.42 ± 0.09 | 0.37 ± 0.08 |

TABLE 9
Effect of atropine sulfate eye drops with different pH
values on NITNI attenuation time of eyes (x̅ ± S, n = 15)

| | Observation time (days) | | |
|---|---|---|---|
| Group | 0 | 7 | 14 |
| Control group | 138.9 ± 9.5 | 143.5 ± 9.5 | 144.3 ± 8.2 |
| pH4.0 | 142.9 ± 10.1 | 128.8 ± 11.3 | 125.7 ± 12.4 |
| pH4.5 | 144.1 ± 11.8 | 117.0 ± 9.3 | 96.2 ± 8.9 |
| pH5.0 | 138.7 ± 11.8 | 116.0 ± 11.5 | 95.6 ± 7.9 |
| pH5.5 | 134.4 ± 11.5 | 112.4 ± 6.7 | 92.1 ± 7.5 |
| pH6.0 | 142.9 ± 11.0 | 127.0 ± 14.1 | 112.3 ± 7.7 |
| pH6.5 | 136.7 ± 12.4 | 136.7 ± 12.4 | 135.4 ± 11.7 |

It could be seen from the data in Table 8 and Table 9 that the pH value had an impact on the effect of atropine in the treatment of NITM. The treatment effect was the best within the pH value ranging from 4.5 to 5.5. When the pH was increased to 6.0 or above, the effect would decrease, suggesting that the pH value of atropine eye drops for the treatment of NITM should preferably range from 4.5 to 5.5.

### Example 4-8: Study on Effect of Atropine Sulfate Eye Drops of Different Prescriptions on NITM (1)

### 1. Experimental Drugs and Preparation Methods Thereof

TABLE A
Prescription compositions of experimental drugs

| Component | Example 4 | Example 5 | Example 6 | Example 7 | Example 8 |
|---|---|---|---|---|---|
| Atropine sulfate | 0.005 g | 0.010 g | 0.020 g | 0.010 g | 0.020 g |
| Hypromellose | — | — | — | 1.000 g | 1.000 g |
| Sodium dihydrogen | 0.250 g | 0.250 g | 0.250 g | 0.250 g | 0.250 g |
| phosphate | | | | | |
| Disodium hydrogen | 0.0025 g | 0.0025 g | 0.0025 g | 0.0025 g | 0.0025 g |
| phosphate | | | | | |
| Disodium edetate | 0.010 g | 0.010 g | 0.010 g | 0.010 g | 0.010 g |
| Sodium chloride | 0.76 g | 0.76 g | 0.76 g | 0.76 g | 0.76 g |
| Benzalkonium | 0.010 g | 0.010 g | 0.010 g | 0.010 g | 0.010 g |
| chloride | | | | | |
| Water for injection, | 100 ml | 100 ml | 100 ml | 100 ml | 100 m1 |
| added to reach a volume of | | | | | |

The preparation method was as follows:

(1) 10 g of 80° C. to 90° C. water for injection was taken, added with a prescribed amount of hypromellose (if any), supplemented with 30° C. or below water for injection to 20 g, stirred for dissolution to obtain a transparent solution for later use;

(2) 50 g of 65° C. to 75° C. water for injection was taken, added with sodium dihydrogen phosphate monohydrate, disodium hydrogen phosphate, sodium chloride, disodium edetate and benzalkonium chloride in prescribed amounts in sequence for dissolution, stood to 30° C. or below and added with a prescribed amount of atropine sulfate and stirred to dissolve;

(3) the hypromellose solution obtained in (1) and the solution obtained in (2) were mixed uniformly;

(4) water was supplemented to the full amount of 100 g, stirred evenly, filtered and sterilized with 0.22 μm filter membrane, and packaged.

They were used for administration in the test groups.

### 2. Experimental Method

90 Myopia patients aged 6 to 18 were selected. The selected patients who had eye diopter of −0.50DS to −5.00DS, astigmatism [symbol] −1.0DC; initial NITM value [symbol] −0.25D; normal intraocular pressure not exceeding 21 mmHg were randomly divided into 6 groups, 15 patients in each group. The 5 test groups were separately dripped with the above atropine eye drops (which were prepared in Examples 4 to 8), and the control group was dripped with 0.9% normal saline; each group was administered once a day, 1 drop per eye each time, and the aforementioned drug or 0.9% normal saline was dripped into conjunctival sac before sleep for consecutive 2 weeks. NITM detection was performed on the 0^{th} day, 7^{th} day, 14^{th} day, and the initial NITM value and NITM attenuation time were recorded.

Optometry was carried out by using a phoropter (non-cycloplegia), according to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again, and the initial NITM was recorded.

### 3. Experimental Results

The results were shown in Table 10 to Table 11 below.

TABLE 10
Effect of atropine sulfate eye drops with different
prescriptions on initial NITM value of eves (x̅ ± S, n = 15)

| | Observation time (days) | | |
|---|---|---|---|
| Group | 0 | 7 | 14 |
| Control group | 0.44 ± 0.10 | 0.42 ± 0.11 | 0.43 ± 0.12 |
| Example 4 | 0.43 ± 0.11 | 0.34 ± 0.08 | 0.33 ± 0.10 |
| Example 5 | 0.41 ± 0.09 | 0.28 ± 0.06 | 0.27 ± 0.06 |
| Example 6 | 0.39 ± 0.07 | 0.20 ± 0.07 | 0.21 ± 0.08 |
| Example 7 | 0.45 ± 0.10 | 0.75 ± 0.06 | 0.25 ± 0.10 |
| Example 8 | 0.46 ± 0.08 | 0.19 ± 0.07 | 0.16 ± 0.05 |

TABLE 11
Effect of atropine sulfate eye drops with different
prescriptions on NITM attenuation time of eyes (x̅ ± S, n = 15)

| | Observation time (days) | | |
|---|---|---|---|
| Group | 0 | 7 | 14 |
| Control group | 143.3 ± 11.8 | 137.0 ± 14.1 | 137.8 ± 11.6 |
| Example 4 | 135.3 ± 12.5 | 121.1 ± 9.1 | 116.1 ± 6.9 |
| Example 5 | 136.7 ± 12.6 | 115.7 ± 9.9 | 108.4 ± 11.4 |
| Example 6 | 142.0 ± 12.0 | 102.4 ± 15.0 | 97.4 ± 10.5 |
| Example 7 | 139.1 ± 11.0 | 112.1 ± 8.7 | 104.0 ± 13.2 |
| Example 8 | 138.0 ± 10.9 | 96.9 ± 9.7 | 93.7 ± 8.9 |

It could be seen from the data in Table 10 and Table 11 that after atropine was prepared as eye drops in concentrations from 0.005% to 0.02%, the atropine eye drops showed a dose-effect relationship in the treatment of NITM. After hypromellose was added in the prescriptions, the therapeutic effects tended to be enhanced, which was more obvious in high-concentration preparations; however, compared with the change of therapeutic concentration, its enhancement effect was weaker. However, it was still suggested that under the same concentration of atropine sulfate, hypromellose was preferably added to the formulation.

### Example 9-13: Study on Effect of Atropine Sulfate Eye Drops of Different Prescriptions on NITM (2)

### 1. Experimental Drugs and Preparation Methods Thereof

TABLE B
Prescription compositions of experimental drugs

| Component | Example 9 | Example 10 | Example 11 | Example 12 | Example 13 |
|---|---|---|---|---|---|
| Atropine sulfate | 0.010 g | 0.020 g | 0.010 g | 0.020 g | 0.010 g |
| Hypromellose | 1.000 g | 1.000 g | 1.000 g | 1.000 g | 1.000 g |
| Propylene glycol | 0.300 g | 0.300 g | — | — | — |
| Sodium chloride | — | — | 0.135 g | 0.135 g | |
| Boric acid | 1.750 g | 1.750 g | 1.750 g | 1.750 g | 2.0 g |
| Disodium edetate | 0.010 g | 0.010 g | 0.010 g | 0.010 g | 0.010 g |
| Benzalkonium | 0.010 g | — | — | 0.010 g | — |
| chloride | | | | | |
| Water for injection, | 100 m1 | 100 m1 | 100 m1 | 100 m1 | 100 m1 |
| added to reach a | | | | | |
| volume of | | | | | |

Preparation Method:

(1) 10 g of 80° C. to 90° C. water for injection was taken, added with a prescribed amount of hypromellose and fully dispersed and swelled, supplemented with 30° C. or below water for injection to 20 g, stirred for dissolution to obtain a transparent solution for later use;

(2) 50 g of 65° C. to 75° C. water for injection was taken, added with boric acid, propylene glycol or sodium chloride (if any), disodium edetate and benzalkonium chloride in prescribed amounts in sequence for dissolution, stood to 30° C. or below and added with a prescribed amount of atropine sulfate and stirred to dissolve;

(3) the hypromellose solution obtained in (1) and the solution obtained in (2) were mixed uniformly:

(4) water was supplemented to the mixed solution obtained in (3) to reach the full amount of 100 g, stirred evenly, filtered and sterilized with 0.22 μm filter membrane, and packaged.

They were used for administration in the test groups.

### 2. Experimental Method

90 Myopia patients aged 6 to 18 were selected. The selected patients who had eye diopter of −0.50DS to −5.00DS, astigmatism [symbol] −1.0DC; initial NITM value [symbol] −0.25D normal intraocular pressure not exceeding 21 mmHg were randomly divided into 6 groups, 15 patients in each group. The 5 test groups were separately dripped with the above atropine eye drops (which were prepared in Examples 9 to 13), and the control group was dripped with 0.9% normal saline, each group was administered once a day, 1 drop per eye each time, and the aforementioned drug or 0.9% normal saline was dripped into conjunctival sac before sleep for consecutive 2 weeks. NITM detection was performed on the 0^{th} day, 7^{th} day, 14^{th} day, and the initial NITM value, NITM attenuation time and pupil diameter before the detection of NITM were recorded.

Optometry was carried out by using a phoropter (non-cycloplegia) and the pupil diameter was measured, according to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again.

### 3. Experimental Results

The results were shown in Table 12 to Table 14 below.

TABLE 12
Effect of atropine sulfate eye drops with different
prescriptions on initial NUM value of eyes (x̅ ± S n = 15)

| | Observation time (days) | | |
|---|---|---|---|
| Group | 0 | 7 | 14 |
| Control group | 0.44 ± 0.14 | 0.45 ± 0.10 | 0.46 ± 0.09 |
| Example 9 | 0.44 ± 0.10 | 0.22 ± 0.07 | 0.21 ± 0.08 |
| Example 10 | 0.42 ± 0.10 | 0.14 ± 0.05 | 0.13 ± 0.07 |
| Example 11 | 0.44 ± 0.12 | 0.25 ± 0.08 | 0.23 ± 0.08 |
| Example 12 | 0.41 ± 0.10 | 0.18 ± 0.07 | 0.15 ± 0.05 |
| Example 13 | 0.46 ± 0.09 | 0.20 ± 0.06 | 0.20 ± 0.07 |

TABLE 13
Effect of atropine sulfate eye drops with different prescriptions on NITM
attenuation time of eyes (x̅ ± S, n = 15)

| | Observation time (days) | | |
|---|---|---|---|
| Group | 0 | 7 | 14 |
| Control group | 137.2 ± 12.6 | 142.7 ± 11.8 | 140.0 ± 11.3 |
| Example 9 | 140.0 ± 12.4 | 107.2.1 ± 12.1 | 98.2 ± 12.2 |
| Example 10 | 136.7 ± 9.3 | 93.8 ± 8.1 | 88.9 ± 8.3 |
| Example 11 | 140.3 ± 12.3 | 113.1 ± 7.7 | 100.3 ± 12.9 |
| Example 12 | 141.3 ± 11.7 | 96.1 ± 9.8 | 91.2 ± 7.7 |
| Example 13 | 142.5 ± 12.9 | 100.7 ± 8.4 | 95.1 ± 8.0 |

TABLE 14
Effect of atropine sulfate eye drops with different prescriptions
on pupil diameter of eyes (x̅ ± S, n = 15)

| | Observation time (days) | | |
|---|---|---|---|
| Group | 0 | 7 | 14 |
| Control group | 3.6 ± 0.9 | 3.6 ± 1.0 | 3.3 ± 0.9 |
| Example 9 | 3.6 ± 0.9 | 4.4 ± 0.7 | 4.1 ± 0.8 |
| Example 10 | 3.8 ± 0.8 | 5.0 ± 1.0 | 4.8 ± 0.9 |
| Example 11 | 3.8 ± 0.9 | 4.3 ± 0.8 | 4.3 ± 0.9 |
| Example 12 | 3.2 ± 0.9 | 5.2 ± 1.0 | 5.0 ± 0.8 |
| Example 13 | 3.4 ± 0.8 | 4.2 ± 0.8 | 3.9 ± 0.6 |

The comparison of the data in Table 12 and Table 13 showed that when boric acid was used as the buffer system, the therapeutic effect of atropine eye drops on NITM was better than that of the phosphate buffer system. When polyol was used as the osmotic pressure regulator, the therapeutic effect was slightly better than that of the inorganic salt osmotic pressure regulator, but the inventors unexpectedly discovered that when only boric acid was used as the pH regulating agent and used to adjust the osmotic pressure at the same time, much better therapeutic effect was achieved.

It could be seen from the data in Table 14 that the boric acid buffer salt system could not only improve the therapeutic effect on NITM, but also had low mydriatic side effect, especially when boric acid was used alone (Example 13), the weakest mydriatic effect was achieved.

### Examples 14-18: Study on Effect of Atropine Sulfate Eye Drops of Different Prescriptions on NITM (3)

### 1. Experimental Drugs and Preparation Methods Thereof

TABLE C
Prescription compositions of experimental drugs

| Com- | Example | | Example | | Example | | Example | | Example | |
|---|---|---|---|---|---|---|---|---|---|---|
| ponent | 14 | | 15 | | 16 | | 17 | | 18 | |
| Atropine | 0.010 | g | 0.010 | g | 0.010 | g | 0.010 | g | 0.010 | g |
| sulfate | | | | | | | | | | |
| Hypro- | 0.800 | g | 0.500 | g | 1.000 | g | 1.200 | g | 1.000 | g |
| mellose | | | | | | | | | | |
| Sodium | 0.02 | g | 0.05 | g | — | | — | | — | |
| hyaluronate | | | | | | | | | | |
| Boric acid | 2.00 | g | 2.00 | g | 1.750 | g | 2.0 | g | 1.750 | g |
| Disodium | 0.005 | g | 0.010 | g | 0.050 | g | 0.010 | g | 0.100 | g |
| edetate | | | | | | | | | | |
| Benzal- | — | | 0.010 | g | — | | 0.010 | g | — | |
| konium | | | | | | | | | | |
| chloride | | | | | | | | | | |
| Hydro- | 4.5 | | 5.0 | | 5.5 | | 5.0 | | 6.0 | |
| chloric | | | | | | | | | | |
| acid or | | | | | | | | | | |
| sodium | | | | | | | | | | |
| hydroxide, | | | | | | | | | | |
| added to | | | | | | | | | | |
| adjust pH | | | | | | | | | | |
| to | | | | | | | | | | |
| Water for | 100 | ml | 100 | ml | 100 | ml | 100 | ml | 100 | ml |

injection,
added to
reach a
volume of

Preparation Method:

(1) 10 g of 80° C. to 90° C. water for injection was taken, added with a prescribed amount of hypromellose or sodium hyaluronate (if any) and fully dispersed and swelled, supplemented with 30° C. or below water for injection to 20 g, stirred for dissolution to obtain a transparent solution for later use;

(2) 50 g of 65° C. to 75° C. water for injection was taken, added with boric acid, disodium edetate and benzalkonium chloride (if any) in prescribed amounts in sequence for dissolution, stood to 30° C. or below and added with a prescribed amount of atropine sulfate and stirred to dissolve;

(3) the solution obtained in (1) and the solution obtained in (2) were mixed uniformly;

(4) water was supplemented to the mixed solution obtained in (3) to reach the full amount of 100 g, stirred evenly, filtered and sterilized with 0.22 μm filter membrane, and packaged.

They were used for administration in the test groups.

### 2. Experimental Method

90 Myopia patients aged 6 to 18 were selected. The selected patients who had eye diopter of −0.50DS to −5.00DS, astigmatism [symbol] −1.0DC; initial NITM value [symbol] −0.25D; normal intraocular pressure not exceeding 21 mmHg were randomly divided into 6 groups, 15 patients in each group. The 5 test groups were separately dripped with the above atropine eye drops (which were prepared in Examples 14 to 18), and the control group was dripped with 0.9% normal saline; each group was administered once a day, 1 drop per eye each time, and the aforementioned drug or 0.9% normal saline was dripped into conjunctival sac before sleep for consecutive 2 weeks. NITM detection was performed on the 0^{th} day, 7^{th} day, 14^{th} day, and the initial NITM value and attenuation time were recorded.

Optometry was carried out by using a phoropter (non-cycloplegia) and the pupil diameter was measured, according to the optometry results, full-correction frame glasses were worn to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again, and the initial NITM was recorded.

### 3. Experimental Results

The results were shown in Table 15 to Table 16 below.

TABLE 15
Effect of atropine sulfate eye drops with different
prescriptions on initial NITM value of eyes (x̅ ± S, n = 15)

| | Observation time (days) | | |
|---|---|---|---|
| Group | 0 | 7 | 14 |
| Control group | 0.46 ± 0.11 | 0.42 ± 0.11 | 0.40 ± 0.09 |
| Example 14 | 0.45 ± 0.11 | 0.26 ± 0.09 | 0.23 ± 0.07 |
| Example 15 | 0.44 ± 0.09 | 0.23 ± 0.07 | 0.20 ± 0.07 |
| Example 16 | 0.43 ± 0.11 | 0.22 ± 0.07 | 0.20 ± 0.08 |
| Example 17 | 0.44 ± 0.10 | 0.23 ± 0.07 | 0.24 ± 0.04 |
| Example 18 | 0.45 ± 0.09 | 0.36 ± 0.09 | 0.34 ± 0.07 |

TABLE 16
Effect of atropine sulfate eye drops with different prescriptions
on NITM attenuation time of eyes (x̅ ± S, n = 15)

| | Observation time (days) | | |
|---|---|---|---|
| Group | 0 | 7 | 14 |
| Control group | 143.3 ± 14.2 | 139.2 ± 11.9 | 141.9 ± 13.2 |
| Example 14 | 137.1 ± 13.6 | 108.7 ± 7.2 | 91.5 ± 10.5 |
| Example 15 | 139.3 ± 9.1 | 106.8 ± 12.1 | 86.8 ± 8.0 |
| Example 16 | 143.1 ± 10.1 | 105.9 ± 12.6 | 83.9 ± 102 |
| Example 17 | 142.6 ± 10.1 | 106.5 ± 15.4 | 92.5 ± 10.6 |
| Example 18 | 138.3 ± 13.6 | 126.4 ± 13.9 | 107.1 ± 13.5 |

The results show that the pharmaceutical compositions of the present invention could effectively treat and/or prevent NITM in patients with myopia. In addition, the results also showed that the effects of the pharmaceutical compositions prepared in Examples 14 to 17 were similar, while the effect of the pharmaceutical composition of Example 18 was relatively poor, so the preferred pH was 6.0 or less.

### Examples 19-23: Study on Effect of Atropine Sulfate Eye Drops of Different Prescriptions on NITM (4)

### 1. Experimental Drugs and Preparation Methods Thereof

The experimental drugs used in Examples 19-23 were exactly the same as those used in the previous Examples 14-18.

They were used for administration in the test group.

### 2. Experimental Method

90 Persons aged 6 to 18 with normal eyesight were selected, who had initial NITM value [symbol] −0.20D; normal intraocular pressure not exceeding 21 mmHg were randomly divided into 6 groups, 15 persons in each group. The 5 test groups were separately dripped with the above atropine eye drops (which were prepared in Examples 14 to 18), and the control group was dripped with 0.9% normal saline; each group was administered once a day, 1 drop per eye each time, and dripped into conjunctival sac before sleep for consecutive 2 weeks. NITM detection was performed on the 0^{th} day 7^{th} day, 14^{th} day, and the initial NITM value and attenuation time were recorded.

Optometry was carried out by using a phoropter (non-cycloplegia) and the pupil diameter was measured, according to the optometry results, full-correction frame glasses were wom to correct the distance vision to the best corrected visual acuity, and the spherical equivalent degree was obtained. A rest for 10 minutes in a complete dark room was taken to relax any possible adjustments. Then, an infrared auto-refractor (WAM-5500, Grand Seiko, Japan) was used, both eyes looked at the 5 m distance mark, the long distance diopter of right eye was measured, and the data was recorded. Then near-work was started, reading was performed with both eyes for the near-work, the reading content was simplified Chinese text of 12pt font, the reading distance was 35 to 40 cm, and the reading time was 1 h. All subjects should pay attention to the text and keep the text clear, and should not be distracted during the procedure. After finishing the near-work, the subject quickly (within 2 s) looked at the distance mark. The long distance diopter of the right eye wearing full-correction frame glasses was measured again, and the initial NITM was recorded.

### 3. Experimental Results

The results were shown in Table 17 to Table 18 below.

TABLE 17
Effect of atropine sulfate eye drops with different prescriptions
on initial NITM value of emmetropic eyes (x̅ ± S, n = 15)

| | Observation time (days) | | |
|---|---|---|---|
| Group | 0 | 7 | 14 |
| Control group | 0.34 ± 0.07 | 0.32 ± 0.08 | 0.31 ± 0.06 |
| Example 14 | 0.31 ± 0.06 | 0.26 ± 0.10 | 0.16 ± 0.06 |
| Example 15 | 0.33 ± 0.06 | 0.24 ± 0.09 | 0.15 ± 0.06 |
| Example 16 | 0.32 ± 0.07 | 0.22 ± 0.09 | 0.15 ± 0.07 |
| Example 17 | 0.33 ± 0.06 | 0.26 ± 0.09 | 0.16 ± 0.07 |
| Example 18 | 0.34 ± 0.06 | 0.29 ± 0.05 | 0.22 ± 0.08 |

TABLE 18
Effect of atropine sulfate eye drops with different prescriptions
on NITM attenuation time of emmetropic eyes (x̅ ± S, n = 15)

| | Group Observation time (days) | | |
|---|---|---|---|
| Group | 0 | 7 | 14 |
| Control group | 70.7 ± 10.2 | 72.3 ± 11.4 | 67.5 ± 13.3 |
| Example 14 | 71.0 ± 13.5 | 51.8 ± 14.6 | 41.4 ± 5.6 |
| Example 15 | 69.5 ± 11.2 | 47.4 ± 10.9 | 38.5 ± 6.3 |
| Example 16 | 65.7 ± 9.6 | 45.3 ± 12.6 | 37.5 ± 5.0 |
| Example 17 | 66.7 ± 8.7 | 49.1 ± 11.2 | 42.0 ± 4.6 |
| Example 18 | 67.7 ± 12.0 | 60.5 ± 11.5 | 55.5 ± 10.1 |

The results showed that the pharmaceutical compositions of the present invention could effectively treat and/or prevent NITM in subjects with normal eyesight. In addition, the results also showed that the effects of the pharmaceutical compositions prepared in Examples 14-17 were similar, and the effect of the pharmaceutical composition of Example 18 was relatively poor, so the preferred pH was 6.0 or less.

Although the specific embodiments of the present invention have been described in detail, those skilled in the art will understand, according to all the teachings that have been disclosed, various modifications and substitutions can be made to those details, and these modifications are all within the protection scope of the present invention. The full scope of the invention is given by the appended claims and any equivalents thereof.

1. A pharmaceutical composition, comprising atropine or a pharmaceutically acceptable salt thereof in an amount of 0.001% to 0.2%, and one or more pharmaceutically acceptable excipients;
wherein,
the pharmaceutical composition has a pH value of 4.0 to 6.5;
the pharmaceutical composition comprises a pH adjusting agent in an amount of 0.05% to 5%, and the pH adjusting agent is one or more selected from a group consisting of sodium dihydrogen phosphate, disodium hydrogen phosphate, citric acid, citrate, boric acid and borate.

2. The pharmaceutical composition according to claim 1, which is an ophthalmic preparation, such as an ophthalmic liquid preparation (e.g., eye drop, eye wash or intraocular injection solution), ophthalmic semi-solid preparation (e.g., eye ointment, ophthalmic cream or ophthalmic gel), or ophthalmic solid preparation (e.g., eye film, eye pill or intraocular insert); optionally, the ophthalmic liquid preparation may be packaged in a solid form, in which a solvent is provided separately, and a solution or suspension is prepared before use.

3. The pharmaceutical composition according to claim 2, wherein the atropine or a pharmaceutically acceptable salt thereof has a concentration or content of 0.001% to 0.1%, preferably 0.005% to 0.05%, more preferably 0.005% to 0.02%, more preferably 0.005% to 0.015%, particularly preferably 0.01%; preferably, the pharmaceutically acceptable salt of atropine is atropine sulfate.

4. The pharmaceutical composition according to claim 1, wherein the pharmaceutical composition has a pH of 4.0 to 6.0, preferably 4.5 to 6.0, more preferably 4.5 to 5.5.

5. The pharmaceutical composition according to claim 1, wherein:
the pH adjusting agent has a content of 0.5% to 5%, more preferably 1% to 3%, further preferably 1.5% to 2.5%, particularly preferably 1.75% to 2.0%;
preferably, the citrate salt is sodium citrate, and/or the borate salt is sodium tetraborate.

6. The pharmaceutical composition according to claim 1, wherein the pharmaceutical composition comprises boric acid in an amount of 0.5% to 3%, 1% to 3%, 1.5% to 2.5%, 1.75% to 2.25% or 1.75% to 2%.

7. The pharmaceutical composition according to claim 1, wherein the pharmaceutical composition further comprises any one or more selected from the following items 1) to 5):
1) a thickener, and the thickener is any one or more selected from a group consisting of hypromellose, sodium carboxymethyl cellulose and sodium hyaluronate; preferably, the thickener has a content of 0.01% to 5%, preferably 0.5% to 3%, more preferably 0.5% to 1.5%, particularly preferably 0.8% to 1.2%;
2) an osmotic pressure regulator, for example, any one or more selected from a group consisting of glycerin, mannitol, propylene glycol, sodium chloride and potassium chloride;
3) a preservative, for example, any one or more selected from a group consisting of benzalkonium chloride, paraben bacteriostatic agents, and polyquaterniums;
4) a stabilizer, for example, any one or more selected from a group consisting of disodium edetate and calcium sodium edetate;
5) an appropriate amount of water.

8. The pharmaceutical composition according to claim 1, wherein the ingredients and contents thereof in the pharmaceutical composition are selected from any one of the following groups (1) to (8):

| (1) | |
|---|---|
| Atropine sulfate | 0.05 to 0.15 parts by weight |
| Hypromellose | 0.5 to 1.5 parts by weight |
| Sodium hyaluronate | 0 to 0.1 parts by weight |
| Boric acid | 1.5 to 2.5 parts by weight |
| Disodium edetate | 0 to 0.1 parts by weight |
| Benzalkonium chloride | 0 to 0.01 parts by weight |

Hydrochloric acid or sodium hydroxide, added to adjust pH to 4.5 to 6.0
Water for injection, added to reach 100 parts by weight;

| (2) | |
|---|---|
| Atropine sulfate | 0.01 parts by weight |
| Hypromellose | 0.5 to 1.2 parts by weight |
| Sodium hyaluronate | 0 to 0.05 parts by weight |
| Boric acid | 1.5 to 2.0 parts by weight |
| Disodium edetate | 0.005 to 0.1 parts by weight |
| Benzalkonium chloride | 0 to 0.01 parts by weight |

Hydrochloric acid or sodium hydroxide, added to adjust pH to 4.5 to 5.5
Water for injection, added to reach 100 parts by weight;

| (3) | |
|---|---|
| Atropine sulfate | 0.01 parts by weight |
| Hypromellose | 0.8 to 1.0 parts by weight |
| Sodium hyaluronate | 0.02 to 0.05 parts by weight |
| Boric acid | 1.75 to 2.0 parts by weight |
| Disodium edetate | 0.01 to 0.05 parts by weight |
| Benzalkonium chloride | 0 to 0.01 parts by weight |

Hydrochloric acid or sodium hydroxide, added to adjust pH to 4.5 to 5.5
Water for injection, added to reach 100 parts by weight;

| (4) | | |
|---|---|---|
| Atropine sulfate | 0.010 | parts by weight |
| Hypromellose | 0.800 | parts by weight |
| Sodium hyaluronate | 0.02 | parts by weight |
| Boric acid | 2.00 | parts by weight |
| Disodium edetate | 0.005 | parts by weight |
| Benzalkonium chloride | 0 | parts by weight |

Hydrochloric acid or sodium hydroxide, added to adjust pH to 4.5
Water for injection, added to reach 100 parts by weight;

| (5) | | |
|---|---|---|
| Atropine sulfate | 0.010 | parts by weight |
| Hypromellose | 0.500 | parts by weight |
| Sodium hyaluronate | 0.05 | parts by weight |
| Boric acid | 2.00 | parts by weight |
| Disodium edetate | 0.010 | parts by weight |
| Benzalkonium chloride | 0.010 | parts by weight |

Hydrochloric acid or sodium hydroxide, added to adjust pH to 5.0
Water for injection, added to reach 100 parts by weight;

| (6) | | |
|---|---|---|
| Atropine sulfate | 0.010 | parts by weight |
| Hypromellose | 1.000 | parts by weight |
| Sodium hyaluronate | 0 | parts by weight |
| Boric acid | 1.750 | parts by weight |
| Disodium edetate | 0.050 | parts by weight |
| Benzalkonium chloride | 0 | parts by weight |

Hydrochloric acid or sodium hydroxide, added to adjust pH to 5.5
Water for injection, added to reach 100 parts by weight;

| (7) | | |
|---|---|---|
| Atropine sulfate | 0.010 | parts by weight |
| Hypromellose | 1.200 | parts by weight |
| Sodium hyaluronate | 0 | parts by weight |
| Boric acid | 2.0 | parts by weight |
| Disodium edetate | 0.010 | parts by weight |
| Benzalkonium chloride | 0.010 | parts by weight |

Hydrochloric acid or sodium hydroxide, added to adjust pH to 5.0
Water for injection, added to reach 100 parts by weight;

| (8) | | |
|---|---|---|
| Atropine sulfate | 0.010 | parts by weight |
| Hypromellose | 1.000 | parts by weight |
| Sodium hyaluronate | 0 | parts by weight |
| Boric acid | 1.750 | parts by weight |
| Disodium edetate | 0.100 | parts by weight |
| Benzalkonium chloride | 0 | parts by weight |

Hydrochloric acid or sodium hydroxide, added to adjust pH to 6.0
Water for injection, added to reach 100 parts by weight.

9.-12. (canceled)

13. A packed medicament product, which comprises a medicament and a product specification;
wherein, the medicament is atropine or a pharmaceutically acceptable salt thereof, or a pharmaceutical composition comprising atropine or a pharmaceutically acceptable salt thereof,
preferably, the pharmaceutically acceptable salt is atropine sulfate;
preferably, the pharmaceutical composition further comprises one or more pharmaceutically acceptable excipients;
preferably, the pharmaceutical composition is the pharmaceutical composition according to claim 1;
preferably, the medicament is an ophthalmic preparation, preferably an eye drop.

14. The packed medicament product according to claim 13, wherein the product specification states that the eye drop is administered once a day or every other day, with 1 to 2 drops each time;
preferably, the product specification also states that the eye drop is dripped into a conjunctival sac.

15. The packed medicament product according to claim 13, wherein the product specification states that the eye drop is administered to a subject who is a myopia patient or a non-myopia patient;
preferably, the product specification states that the eye drop is administered to a subject who is a person using eyes in near distance;
preferably, the product specification states that the eye drop is administered to a subject who is a myopia patient or non-myopia patient using eyes in near distance;
preferably, the product specification states that the eye drop is administered to a subject who is a person susceptible to myopia;
preferably, the product specification states that the eye drop is administered to a subject who is a person aged 6 to 18, such as a myopia patient aged 6 to 18 or a non-myopia patient aged 6 to 18.

16.-19. (canceled)

20. A method of treating and/or preventing NITM, comprising a step of administering an effective amount of a medicament to a subject in need thereof, wherein the medicament is atropine or a pharmaceutically acceptable salt thereof, or a pharmaceutical composition containing atropine or a pharmaceutically acceptable salt thereof;
preferably, the pharmaceutically acceptable salt is atropine sulfate;
preferably, the pharmaceutical composition further comprises one or more pharmaceutically acceptable excipients;
preferably, the pharmaceutical composition is the pharmaceutical composition according to claim 1.

21. The method for treating and/or preventing NITM according to claim 20, wherein the pharmaceutical composition is an ophthalmic preparation, preferably an eye drop.

22. The method for treating and/or preventing NITM according to claim 21, wherein the eye drop is administered once a day or every other day, with 1 to 2 drops each time; preferably before sleep;
preferably, the eye drop is dripped into a conjunctival sac.

23. The method for treating and/or preventing NITM according to claim 21, wherein the eye drop is administered to a subject who is a myopia patient or a non-myopia patient;
preferably, the eye drop is administered to a subject who is a person using eyes in near distance;
preferably, the eye drop is administered to a subject who is a myopia patient or non-myopia patient using eyes in near distance;
preferably, the eye drop is administered to a subject who is a person susceptible to myopia, such as a person aged 6 to 18;
preferably, the person susceptible to myopia is a myopia patient or a non-myopia patient.
